(19) United States
(12) Patent Application Publication
(10) Pub. No.: US 2023/0364014 A1
(43) Pub. Date: Nov. 16, 2023

(54) SELF-ASSEMBLED GELS FOR CONTROLLED DELIVERY OF BIOLOGICS AND METHODS OF MAKING THEREOF

(71) Applicant: Alivio Therapeutics, Inc., Boston, MA (US)

(72) Inventors: Gregory T. Zugates, Chelmsford, MA (US); Julia Wang, Forest Hills, NY (US); Derek G. van der Poll, Medford, MA (US); Dominick J. Blasioli, Chelmsford, MA (US)

(73) Assignee: Alivio Therapeutics, Inc., Boston, MA (US)

(21) Appl. No.: 18/324,946

(22) Filed: May 26, 2023

Related U.S. Application Data
(62) Division of application No. 16/375,285, filed on Apr. 4, 2019, now abandoned.
(60) Provisional application No. 62/652,548, filed on Apr. 4, 2018.

(51) Int. Cl. A61K 9/00; A61K 47/22; A61K 47/14; A61K 9/06; A61K 9/16; A61K 9/51; A61K 9/14; C07K 16/24

(52) U.S. Cl. CPC A61K 9/006; A61K 47/22; A61K 47/14; A61K 9/06; A61K 9/1682; A61K 9/5123; A61K 9/5192; A61K 9/1617; A61K 9/0031; A61K 9/145; A61K 9/0043; C07K 16/241; A61K 9/0019; A61K 9/0034; A61K 9/1274

(57) ABSTRACT

Gels are formed based on generally recognized as safe (GRAS) low molecular weight amphiphilic molecules in a self-assembly process. Therapeutic or prophylactic agents, such as biological macromolecules, are loaded without exposure to temperatures and/or organic solvents which can degrade or destroy the biologic agents and/or their activity. The resulting self-assembled gel composition contains microstructures having pores and aqueous domains at their interior, rendering them permeable to hydrophilic and hydrophobic molecules. This permeability allows sequestration of the biological macromolecules. Once sequestered, the electrostatic, hydrophobic-hydrophobic etc. interactions between the biological macromolecules and the amphiphilic gelators keep the labile payload encapsulated with high stability until the microstructures are degraded.

### CROSS-REFERENCE TO RELATED APPLICATIONS

This application is a divisional of U.S. patent application Ser. No. 16/375,285 entitled “SELF-ASSEMBLED GELS FOR CONTROLLED DELIVERY OF BIOLOGICS AND METHODS OF MAKING THEREOF” filed Apr. 4, 2019, which claims the benefit of priority to U.S. Provisional Patent Application No. 62/652,548 “entitled SELF-ASSEMBLED GELS FOR CONTROLLED DELIVERY OF BIOLOGICS AND METHODS OF MAKING THEREOF” filed Apr. 4, 2018, both of which are incorporated by reference herein.

### FIELD OF THE INVENTION

This is generally in the field of controlled delivery of biologic, therapeutic, or prophylactic agents, and more particularly, relates to responsive delivery from self-assembled gels that do not compromise the stability and/or activity of encapsulated/entrapped labile therapeutic or prophylactic agents.

### BACKGROUND OF THE INVENTION

Self-assembling gels which are stable in vivo for drug delivery are described in US 2017/0000888. Self-assembly to form molecularly defined, high-ordered structures largely relies on non-covalent interactions. Structures formed from self-assembly are capable of entrapping molecules in solution during the assembly process. These can be administered in the form of gels, dried and rehydrated to form gels, or mechanically broken up into gel particles, which can be injected for delivery of hydrophobic and hydrophilic agents. Most self-assembled gel are formed from amphiphilic compounds which in theory may spontaneous assemble due to hydrophilic-hydrophobic interactions.

Heating in excess of 37-40° C. and/or addition of organic solvent is generally necessary to homogeneously disperse these amphiphilic agents in a medium, such that upon cooling, the amphiphilic agents assemble into ordered nano and micro structures, which can then form a self-supporting gel is formed. The gel is useful for drug delivery, as a reservoir for controlled release of drug agents, and may possess desirable biochemical and mechanical properties as scaffold for tissue repair.

Many therapeutic, prophylactic or biologically active agents, such as biologics, are sensitive to heat and/or organic solvents. Nucleic acids, small compounds, peptide, and other biologically derived components can be labile to heat and/or exposure to certain types of solvents. These agents often lose activity when dissolved into organic solvent and/or heated to over body temperature.

Therefore, it is an object of the present invention to provide a self-assembled gel composition and a process for loading high levels of agents therein with limited to no exposure to heating above body temperature or organic solvent.

It is another object of the present invention to provide a self-assembled gel composition and a process for loading agents wherein the agents have limited to no exposure to organic solvents which can degrade and/or destroy the activity of labile agents, especially biological agents.

It is yet another object of the present invention to provide a self-assembled gel composition that maintains the activity of labile entrapped and/or encapsulated agents upon controlled release.

### SUMMARY OF THE INVENTION

Controlled release hydrogels containing nanostructures formed by self-assembly of amphiphilic compounds having labile therapeutic or prophylactic agents, such as biologics, encapsulated and/or entrapped therein have been formulated using a method so that the labile agents retain activity. Gels are formed based on generally recognized as safe (GRAS) low molecular weight amphiphilic molecules in a self-assembly process. Therapeutic or prophylactic agents, such as biological macromolecules, are loaded without exposure to temperatures and/or organic solvents which can degrade or destroy the biologic agents and/or their activity. The resulting self-assembled gel composition contains nano and/or microstructures having pores and aqueous domains at their interior, rendering them permeable to hydrophilic and hydrophobic molecules. This permeability allows sequestration of the biological macromolecules with the structures. Once sequestered, the electrostatic, hydrophobic-hydrophobic etc. interactions between the biological macromolecules and the amphiphilic gelators keep the labile payload encapsulated with high stability until the structures are degraded.

Release can be regulated as a function of levels of enzymes elevated due to disease severity, through the use of enzyme cleavable linkages between the drug and gelator self-assembling to form the gels. As these levels decrease, the amount of release decreases, due to decreased enzyme cleavage. In contrast to previous methods using organic solvent and elevated temperature to form self-assembled gels encapsulating agent, process conditions have been developed to incorporate agent after the hydrogel formation process. This is a non-trivial way to “encapsulate” an agent in a pre-formed, structured hydrogel. It unexpectedly leads to high drug loading, preserves gel properties, and avoids high solvent/temperature conditions and organic solvent during drug loading, which is important for labile drugs such as many biological macromolecules like antibodies and nucleic acid. For some drugs such as lidocaine, the loading mechanism can be based on electrostatic interactions between the anionic amphiphile head group and the cationic drug, as shown by adding high salt concentrations to break these interactions and release the drug. For other drugs such as some antibodies, the interaction can occur between the drug and the lipophilic tails of the amphiphiles, which can be broken by adding a competing surfactant to release the drug. This last result was unexpected since the lipophilic regions are self-assembled/ordered and buried in the hydrogel. Presumably, these regions would be inaccessible to drug binding/loading, but it was found that it can be done, especially with something as large as an antibody (150 kDa).

The formulations can be provided in the form of gels, lyophilized for administration in dried form which re-hydrate at the site of administration or which is hydrated for administration, disrupted into particles or dispersions, or co-administered with one or more additional therapeutic or prophylactic agents. The amphiphilic compounds are dissolved in an aqueous solvent with mechanical mixing, optionally with heat and/or organic solvent with heating, which is then diluted into aqueous solution. The gel forms as the mixture cools. The gel will typically be filtered, centrifuged, dried or washed to remove the initial solvent so that no detectable amount or only a small residue of organic solvent, (i.e., less than about 5%, 4%, 3%, 2%, 1%, 0.9%, 0.5%, or 0.1% by weight of the resulting gel), if any, is present in the final gel formulation.

Formed hydrogels contain a high loading of therapeutic or prophylactic labile agents. The gel is self-supporting, i.e., stable to inversion at room temperature. Encapsulated agents can maintain at least 50%, 60%, 70%, 80%, or 90% or greater of their activity or intrinsic structural configurations in the self-assembled gel for at least 1 day, 2 days, 3 days, 1 week, 2 weeks, 1 month, or greater in refrigeration, ambient temperature, and/or at 37° C., depending on the agent and temperature at which it is stored. Generally increasing the concentration of gelators increases the encapsulation efficiency of the encapsulated and/or entrapped agents present in the self-assembled gel.

The gels are formed by self-assembly of generally recognized as safe (GRAS), or molecules complying with the requirements of the U.S. Food and Drug Administration for GRAS ingredients, low molecular weight amphiphilic molecules at concentrations of generally at least about 3, 4, 5, 6, 7, 8, 9, 10, 11, 12, 13, 14, 15, 16, 17, 18, 19, or 20 wt/vol %. During gel formation, organic solvent may be used initially to dissolve the gelator, for examples, in an amount of about 15%, 16%, 17%, 18%, 19%, 20%, 21%, 22%, 23%, 24%, 25%, 35%, 40%, 45%, or 50% in volume of the gelation medium. Depending on the types of gelators and combination solvents in the gelation medium, a minimal volume percentage of organic solvent can be required to insure the gelator forms a homogenous solution, thereby forming a gel which is stable to inversion when cooled and inverted at room temperature. Too little organic solvent may result in no gelation (i.e., flowable mass or precipitates of the gelators) or solidification/hardening of the gelators, preventing gelation from happening once water or an aqueous solution is added. Too much organic solvent may also prevent gelation from occurring, or damage labile biological agents to be encapsulated if not sufficiently removed from the gel and nanostructures following gel formation. The organic solvent used in forming the self-assembled gel is removed or substantially removed to a level where the residual amount is within the stated limit of pharmaceutical products by the U.S. Food and Drug Administration (FDA). Drying, solvent exchange, or lyophilization may be used to remove excess organic solvent.

The self-assembled gel is loaded with therapeutic or prophylactic agents, by suspending the gel, which is free or substantially free of organic solvent(s), at a temperature of 37° C. or less, in an aqueous environment, such as a buffer, and mixing the resulting suspension with an aqueous mixture containing one or more agents. In some instances, the self-assembled gel which is free or substantially free of organic solvent(s), may be homogenized, sonicated, or otherwise dispersed to first form particles (i.e, nano- or micro-particles, or a combination thereof) suspended in an aqueous environment, such as a buffer, and mixing the resulting suspension with an aqueous mixture containing one or more agents in order to encapsulate and/or entrap the agents in the gel particles and nanostructures therein.

The self-assembled gel loaded with one or more agents may be suspended in a pharmaceutically acceptable carrier for administration. The self-assembled gel when homogenized, sonicated, or otherwise dispersed as particles may be dried, suspended, or administered in gel. The self-assembled gel, its suspension formulation, or particle formulation may also be incorporated into a bandage, wound dressing, patch, or in a syringe or catheter.

The self-assembled gel, its suspension formulation, or particle formulation, is administered to deliver an effective dosage of the therapeutic or prophylactic agent(s) to alleviate, prevent, or treat one or more symptoms of a disease or disorder.

### BRIEF DESCRIPTION OF THE DRAWINGS

FIG. 1 is a graph showing loading and release profiles of infliximab (IFX) and adalimumab (ADA) loaded ascorbyl palmitate (AP) gels, where the x-axis shows time (hours) and the y-axis shows percent release.

FIG. 2 is a bar graph showing antibody activity of infliximab (IFX) and adalimumab (ADA) released from ascorbyl palmitate (AP) gels which are active against TNF-α in the L929 viability assay (1 ng/mL TNF-α in 2% serum.

FIG. 3 is a graph showing percent loading (y-axis) of infliximab (IFX) into ascorbyl palmitate (AP) fibers over time (x-axis), as determined by HPLC.

FIG. 4 is a non-limiting representation showing the loading dependence of an agent as a function of pH and/or charge of the agent into ascorbyl palmitate (AP) microparticle suspensions.

FIG. 5 is a bar graph showing the encapsulation efficiencies of infliximab (IFX) and adalimumab (ADA) on interaction with ascorbyl palmitate (AP) self-assembled gels versus non-self-assembled AP powder suspensions.

### DETAILED DESCRIPTION OF THE INVENTION

### I. Definitions

The term “gelators” refer to molecules that can self-assemble through non-covalent interactions, such as hydrogen-bonding, van der Waals interactions, hydrophobic interactions, ionic interactions, pi-pi stacking, or combinations thereof, in one or more solvents. The gelators can form a gel by rigidifying the solvent through, for example, capillary forces. Gelators can include hydrogelators (e.g., gelators that form hydrogels) and organo-gelators (e.g., gelators that form organo-gels). In some embodiments, gelators can form both hydrogels and organo-gels.

The term “self-assembling” refers to the capability of molecules to spontaneous assemble, or organize, to form a higher ordered structure such as hydrogel in a suitable environment.

The term “hydrogel” refers to three-dimensional (3-D) networks of molecules covalently (e.g., polymeric hydrogels) or non-covalently (e.g., self-assembled hydrogels) held together where water is the major component. Gels can be formed via self-assembly of gelators or via chemical crosslinking of gelators. Water-based gelators can be used to form hydrogels.

The term “co-assembly”, refers to the process of spontaneous assembly, or organization of at least two different types of molecules to form a high ordered structure such as hydrogel in a suitable environment, where molecules in the structure are generally organized in an ordered manner

The term “organic solvent” refers to any carbon-containing substance that, in its liquid phase, is capable of dissolving a solid substance. Exemplary organic solvents commonly used in organic chemistry include toluene, tetrahydrofuran, acetone, dichloromethane, and hexane.

The term “water-miscible” refers to a solvent that mixes with water, in all proportions, to form a single homogenous liquid phase. This includes solvents like dimethyl sulfoxide (DMSO), tetrahydrofuran, acetone, ethanol, methanol, and dioxane, but generally excludes solvents such as hexane, oils, and ether. It also excludes solvents that have some, very limited miscibility or solubility in water such as ethyl acetate and dichloromethane, which are practically considered immiscible.

The term “percent (%) encapsulated” or “encapsulation percentage” is generally calculated as % encapsulated=weight of encapsulated agent(s)÷weight of total of agent(s) (encapsulated+unencapsulated)×100%.

The term “drug loading efficiency (w/w)” refers to weight drug/(weight drug plus weight amphiphile).

The term “gel weight percent (w/v)” refers to the total mass of gelator(s) as a percentage of total solvent volume (i.e., organic solvent(s)+water for hydrogels).

The term “pharmaceutically acceptable,” as used herein, refers to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the U.S. Food and Drug Administration.

The terms “biocompatible” and “biologically compatible,” as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory or immune response when administered to a patient.

The term “hydrophilic,” as used herein, refers to the property of having affinity for water. For example, hydrophilic polymers (or hydrophilic polymer segments) are polymers (or polymer segments) which are primarily soluble in aqueous solutions and/or have a tendency to absorb water. In general, the more hydrophilic a polymer is, the more that polymer tends to dissolve in, mix with, or be wetted by water.

The term “hydrophobic,” as used herein, refers to the property of lacking affinity for or repelling water. For example, the more hydrophobic a polymer (or polymer segment), the more that polymer (or polymer segment) tends to not dissolve in, not mix with, or not be wetted by water.

The term “surfactant” as used herein refers to an agent that lowers the surface tension of a liquid.

The term “therapeutic agent” refers to an agent that can be administered to prevent or treat one or more symptoms of a disease or disorder. Therapeutic agents can be nucleic acids or analogs thereof, a small molecule (molecular weight of less than 2000 Daltons, more typically less than 1000 Daltons), peptidomimetic, protein, or peptide, carbohydrate or sugar, lipid, or a combination thereof. In some embodiments, cells or cellular materials may be used as therapeutic agents.

The term “treating” or “preventing” a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain.

The term “therapeutically effective amount” refers to an amount of a therapeutic or prophylactic agent, such as a biologic agent, that, when incorporated into and/or onto the self-assembled gel composition, produces some desired effect at a reasonable benefit/risk ratio applicable to any treatment. The effective amount may vary depending on such factors as the disease or condition being treated, the particular formulation being administered, the size of the subject, or the severity of the disease or condition.

The terms “incorporated,” “encapsulated” and “entrapped” refers to incorporating and/or encapsulating and/or entrapping therapeutic or prophylactic agent(s) into in a gel composition or the nanostructures formed therein, regardless of the manner by which the therapeutic or prophylactic agent is incorporated, encapsulated, and/or entrapped.

“GRAS” is an acronym for the phrase Generally Recognized as Safe. Under sections 201(s) and 409 of the Federal Food, Drug, and Cosmetic Act (the Act), any substance that is intentionally added to food is a food additive, that is subject to premarket review and approval by FDA, unless the substance is generally recognized, among qualified experts, as having been adequately shown to be safe under the conditions of its intended use, or unless the use of the substance is otherwise excepted from the definition of a food additive. Under sections 201(s) and 409 of the Act, and FDA's implementing regulations in 21 CFR 170.3 and 21 CFR 170.30, the use of a food substance may be GRAS either through scientific procedures or, for a substance used in food before 1958, through experience based on common use in food Under 21 CFR 170.30(b), general recognition of safety through scientific procedures requires the same quantity and quality of scientific evidence as is required to obtain approval of the substance as a food additive. General recognition of safety through scientific procedures is based upon the application of generally available and accepted scientific data, information, or methods, which ordinarily are published, as well as the application of scientific principles, and may be corroborated by the application of unpublished scientific data, information, or methods. The database of compounds meeting the requirements defined by 21 CFR is found in Title 21: Food and Drugs, Part 184.

Numerical ranges include, but are not limited to, ranges of temperatures, ranges of weight concentrations, ranges of molecular weights, ranges of integers, and ranges of times, etc. The ranges include sub-ranges and combinations of sub-ranges encompassed therein. Use of the term “about” is intended to describe values either above or below the stated value, which the term “about” modifies, in a range of approx. +/−10%; in other instances the values may range in value either above or below the stated value in a range of approx. +/−5%. When the term “about” is used before a range of numbers (i.e., about 1-5) or before a series of numbers (i.e., about 1, 2, 3, 4, etc.) it is intended to modify both ends of the range of numbers or each of the numbers in the series, unless specified otherwise.

### II. Self-Assembled Gel

1. Gelators

Amphiphilic gelators meeting the requirements for the U.S. Food and Drug Administrations list of Generally Required as Safed (“GRAS”) (jointly referred to herein as “GRAS gelators”) which are suitable for self-assembly to form a gel are generally less than 2,500 Da, and may preferably be enzyme-cleavable. The GRAS amphiphile gelators self-assemble into gels formed from and including micro-/nano-structures (e.g., lamellar, micellar, vesicular, and/or fibrous structures).

Preferred GRAS amphiphile gelators include ascorbyl alkanoate, sorbitan alkanoate, triglycerol monoalkanoate, sucrose alkanoate, glycocholic acid, or any combination thereof. In some embodiments, the GRAS amphiphile gelators include ascorbyl palmitate, sorbitan monostearate, triglycerol monopalmitate, sucrose palmitate, or glycocholic acid.

The alkanoate can include a hydrophobic C₁-C₂₂ alkyl (e.g., acetyl, ethyl, propyl, butyl, pentyl, caprylyl, capryl, lauryl, myristyl, palmityl, stearyl, arachidyl, or behenyl) bonded via a labile linkage (e.g., an ester, a carbamate, a thioester and an amide linkage) to an ascorbyl, sorbitan, triglycerol, or sucrose molecule. For example, the ascorbyl alkanoate can be ascorbyl palmitate, ascorbyl decanoate, ascorbyl laurate, ascorbyl caprylate, ascorbyl myristate, ascorbyl oleate, or any combination thereof. The sorbitan alkanoate can be sorbitan monostearate, sorbitan decanoate, sorbitan laurate, sorbitan caprylate, sorbitan myristate, sorbitan oleate, or any combination thereof. The triglycerol monoalkanoate can include triglycerol monopalmitate, triglycerol monodecanoate, triglycerol monolaurate, triglycerol monocaprylate, triglycerol monomyristate, triglycerol monostearate, triglycerol monooleate, or any combination thereof. The sucrose alkanoate can include sucrose palmitate, sucrose decanoate, sucrose laurate, sucrose caprylate, sucrose myristate, sucrose oleate, or any combination thereof.

Representative low molecular weight GRAS amphiphilic gelators include vitamin precursors such as ascorbyl palmitate (vitamin C precursor), retinyl acetate (vitamin A precursor), and alpha-tocopherol acetate (vitamin E precursor).

In some forms, a GRAS amphiphile gelator is formed by synthetically conjugating one or more saturated or unsaturated hydrocarbon chains having C₁ to C₃₀ groups with a low molecular weight, generally hydrophilic compound, through esterification or a carbamate, anhydride, and/or amide linkage. The range C₁ to C₃₀ includes C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉ etc. up to C₃₀ as wells as ranges falling within C₁ to C₃₀, for example, C₁ to C₂₉, C₂ to C₃₀, C₃ to C₂₈, etc.

In some embodiments, alpha tocopherol acetate, retinyl acetate, retinyl palmitate, or a combination thereof, can co-assemble with the gelators.

In some embodiments, to form a viscous gel stable to inversion (e.g., resist flow when inverted at room temperature, approximately 25° C.), greater than 3%, 4%, 5% (wt/vol) or more gelators are completely dissolved in a liquid medium. The gels can include, independently, from about four, from about five, from about 10, or from about 15) to about 40 percent (to about 40, to about 30, to about 20, to about 15, to about 10, to five) of GRAS amphiphile gelators by weight per volume.

In some forms, the self-assembled gel compositions include an enzyme-cleavable, generally recognized as safe (GRAS) first gelator having a molecular weight of 2500 or less and a non-independent second gelator that is also a GRAS agent. Non-independent gelators do not form self-supporting gel at the concentration that would typically form self-supporting gel if combined with an enzyme-cleavable GRAS gelator. Exemplary non-independent second gelators include alpha tocopherol acetate, retinyl acetate, and retinyl palmitate. The non-independent gelators co-assemble with the GRAS first gelators to form the self-assembled gels.

The gels can include, independently, from about three to a maximum of 30-40 percent, more preferably about 4% to 10% by weight gelator per volume of gel. Above 30-40% the gel will begin to precipitate out of solution or become less injectable.

2. Gelation Medium

The liquid medium for the gelators to form self-assembled gel generally includes an aqueous solution or a two-solvent system of an organic solvent and water (or an aqueous salt solution) or an aqueous-organic mixture solvent system. Following gelation the organic solvent(s) are substantially removed (i.e. less than about 5%, 4%, 3%, 2%, 1%, 0.5%, or 0.1% or less of organic solvent(s) by weight in the resulting gel).

In one embodiment, a GRAS gelator is mixed and/or dissolved to homogeneity in an aqueous solution, preferably with strong mechanical mixing and/or heating. In another embodiment, a co-solvent medium including both water (or an aqueous buffer or salt solution) and a water-miscible organic solvent, is used to form a gelation solution.

Alternatively, the GRAS gelator can be dissolved initially in an organic solvent to form a solution with the GRAS gelator as the solute (termed “gelator solution”) and water (or an aqueous buffer or salt solution) can be added subsequently to form the gelation medium.

Organic solvent(s) used in the gelation medium can be selected based on the solubility of gelators therein, its polarity, hydrophobicity, water-miscibility, and in some cases the acidity. Suitable organic solvents include water-miscible solvent or solvent that has an appreciable water solubility (e.g., greater than 5 g/100 g water), e.g., DMSO, dipropylene glycol, propylene glycol, hexyl butyrate, glycerol, acetone, dimethylformamide (DMF), tetrahydrofuran, dioxane, acetonitrile, alcohol such as ethanol, methanol or isopropyl alcohol, as well as low molecular weight polyethylene glycol (e.g., 1 kD PEG which melts at 37° C.). In other forms, the self-assembled gel compositions can include a polar or non-polar solvent, such as water, benzene, toluene, carbon tetrachloride, acetonitrile, glycerol, 1,4-dioxane, dimethyl sulfoxide, ethylene glycol, methanol, chloroform, hexane, acetone, N, N′-dimethyl formamide, ethanol, isopropyl alcohol, butyl alcohol, pentyl alcohol, tetrahydrofuran, xylene, mesitylene, and/or any combination thereof. Organic solvents for gelation include dimethyl sulfoxide (DMSO), dipropylene glycol, propylene glycol, hexyl butyrate, glycerol, acetone, dimethylformamide, tetrahydrofuran, dioxane, acetonitrile, ethanol, and methanol. Another class of organic solvents, fatty alcohols or long-chain alcohols, are usually high-molecular-weight, straight-chain primary alcohols, but can also range from as few as 4-6 carbons to as many as 22-26, derived from natural fats and oils. Some commercially important fatty alcohols are lauryl, stearyl, and oleyl alcohols. Some are unsaturated and some are branched.

The aqueous solvent is typically water which may be sterilized and selected from distilled water, de-ionized water, pure or ultrapure water. In some instances, the second solvent is an aqueous solution such as saline, other physiologically acceptable aqueous solutions containing salts and/or buffers, such as phosphate buffered saline (PBS), Ringer's solution, and isotonic sodium chloride, or any other aqueous solution acceptable for administration to a subject, such as an animal or human. The amounts of aqueous solvent, such as water, is typically based on the amounts of the first organic solvent used wherein the selected total volume or weight percentage of organic solvent(s) determined the volume or weight percentage of the water or aqueous solution (i.e., if 30 v/v % of organic solvent then 70 v/v % water).

In some instances, the amount of an organic solvent is no more than 1:1, 1:2, 1:3, 1:4, 1:5, 1:6, 1:7, 1:8, 1:9, 1:10, or less in volume compared to the volume of an aqueous solution (e.g., water, aqueous buffer, aqueous salt solution, optionally containing one or more additional agents). That is, the volume amount of an organic solvent in the total amount of liquid as used in forming a homogenous gel is generally less than about 50%, 33%, 25%, 20%, 17%, 14%, 12.5%, 11%, 10%, or 9%, and significantly less, typically less than 1%, for particles. Typical ranges are as high as 1:1 and as low as 1:5.

Gelation may require heating the gelation medium to temperatures ranging from between about 30-100° C., about 40-100° C., about 50-100° C., about 60-100° C., about 70-100° C., about 90-100° C., about 30-90° C., about 40-90° C., about 50-90° C., about 60-90° C., about 70-90° C., about 80-90° C., about 40-80° C., about 50-80° C., about 60-80° C., about 70-80° C., about 30-70° C., about 40-70° C., about 50-70° C., about 60-70° C., about 30-60° C., about 40-60° C., about 50-60° C., about 30-50° C., or about 40-50° C. In some embodiments, heating is carried out in the temperature range of between about 60-80° C. In some embodiments, the heating is carried out at about 80° C.

In some instances, no heating is needed, or, if necessary, heating to about body temperature (37° C.) generates a homogeneous self-supporting gel that is stable to inversion. In all cases, the gelation medium is heated to complete dissolution, followed by cooling to about 37° C. or room temperature around 20° C.-25° C.

Gelation can take place with or without heating. When heated, gelation takes place as the heated gelation solution is cooled. Leaving the gel on a stable surface for about one to two hours at room temperature results in a consistent self-supporting gel. Self-supporting gel comprises orderly assembled micro- or nano-structures with minimal precipitates. This is generally confirmed using optical or electron microscopy.

Gelators and solvents are selected at an appropriate gelator concentration and appropriate volume and ratio of the aqueous-organic mixture solvent system, or both, to form self-supporting gel. Preferably, the gelator solution should not solidify or precipitate before the addition of an aqueous solution. Increasing the amount of the organic solvent or reducing the concentration of gelators in the organic solvent may prevent solidification of the gelator solution. When the gelator solution (in an organic solvent) is mixed with the aqueous solution, a self-supporting gel stable to inversion is formed, (following heating if necessary), rather than flowable mass/aggregates.

Following formation of self-supporting gels, the organic solvent in the gel may be removed to a residual level suitable for pharmaceutical applications. In some instances, following gelation, the organic solvent(s) are removed entirely or substantially removed (i.e. less than about 5%, 4%, 3%, 2%, 1%, 0.5%, or 0.1% or less of organic solvent(s) by weight in the resulting gel). One or more purification techniques such as dialysis, centrifugation, filtration, drying, solvent exchange, or lyophilization, can be used to remove organic solvent(s). Residual organic solvent is within the stated limit of pharmaceutical products by the U.S. Food and Drug Administration (FDA) or below the acceptance criteria by U.S. Pharmacopeia Convention, International Conference on Harmonization guidance. For example, dicloromethane is below 600 ppm, methanol below 3,000 ppm, chloroform below 60 ppm; and within the limit by GMP or other quality based requirements.

3. Therapeutic and/or Prophylactic Agents

Therapeutic and/or prophylactic agents, such as biologic agent(s), may be physically entrapped, encapsulated, and/or non-covalently associated with the nanostructures in the gels described above. In the preferred embodiment, they are incorporated into the assembled ordered lamellar, vesicular, and/or nanofibrous structures of the gel composition or positioned on the surface of the assembled structures.

The agent(s) is physically entrapped, encapsulated, and/or non-covalently associated with the nanostructures of the self-assembled gels by forming the gels first. Suspending the gels in an aqueous medium, such as a buffer, where the gel is optionally first broken to form particles (i.e., nano- and/or microparticles) and then mixing the resulting gel particle suspension with a second suspension containing one or more therapeutic or prophylactic agent(s) in order to encapsulate and/or entrap the agent(s) in the gel particles and nanostructures therein.

It is believed that by first initiating gel formation or by forming the gel without loading of agents and then subsequently loading (i.e., encapsulating and/or entrapping) the agent(s) into the self-assembled gel (in bulk or broken into particles thereof), it is possible to preserve the properties of the gel, as opposed to forming the gel in combination with the agent(s) in a single step.

Agent loading level has been shown to be time-dependent and the loading levels thereof may be controlled and/or optimized as a function of the loading/incubation time after mixing the gel suspensions with the second suspension containing one or more biologic therapeutic or prophylactic agent(s). In some instances, the loading/incubation time may be selected from a period of time ranging from between about 0.1 and 48 hours, 0.1 and 36 hours, 0.1 and 24 hours, 0.1 and 20 hours, 0.1 and 15 hours, 0.1 and 10 hours, 0.1 and 5 hours, 0.1 and 1 hours, or ranges therein. In some other instances, the loading/incubation time may be selected from a period of time of at least about 0.1 hours, 0.2 hours, 0.3 hours, 0.4 hours, 0.5 hours, 0.6 hours, 0.7 hours, 0.8 hours, 0.9 hours, 1 hours, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or greater.

The highest encapsulation efficiencies can be as high as 100%, but are typically about 70% up to about 95%. Drug loading (wt/wt) is up to about 50 wt/wt %. A preferred range of drug loading is up to 20% w/w, typically with a maximum of about 30% w/w.

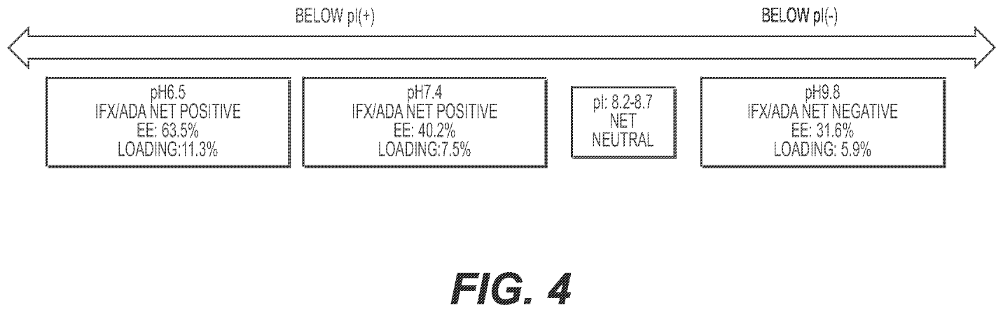

Agent loading level can also be pH and/or charge-dependent and loading of the agent(s) may be controlled and/or optimized based using the pKa and isoelectric (pI) point of the agent. As shown in FIG. 4, agent loading and encapsulation efficiencies are highest at a pH above the pKa of ascorbyl palmitate (AP) but below the pI of IFX or ADA, indicating that electrostatic interactions are a contributing mechanism. Accordingly, agent loading and encapsulation efficiency may be optimized/maximized by loading gel suspensions at a pH above the pKa of the gelator(s) and a pH which is below the isoelectric (pI) point of the agent(s) which are being loaded onto the gel. The pKa and pI of the gelators(s) and agent(s) are either known art known values or may be determined using known techniques Based on the pKa of AP of 4.4 and the pI for infliximab and adalimumab of between 8.2 and 8.7, a pH range between 4.4 and 8.7 is preferred.

Suitable biologic agents include monoclonal antibodies (mAbs), polyclonal antibodies, immunoglobulin, and antigen binding fragments thereof), growth factors (e.g., recombinant human growth factors), antigens, interferons, cytokines, hormones, and other proteins, amino acids, and peptides such as insulin, and combinations thereof, typically those that loose structural integrity, binding ability and/or biological activity if exposed to extensive mixing and/or heating to about 37°. In some instances, the biologic agents are monoclonal antibodies (mAb) such as infliximab (REMICADE®), adalimumab (HUMIRA®), or combinations thereof.

Other antibodies known in the art include, but are not limited to, those discussed in Kaplon H et al., MAbs. 10(2):183-203 (2018). Exemplary antibodies include lanadelumab, crizanlizumab, ravulizumab, eptinezumab, risankizumab, satralizumab, brolucizumab, PRO140, sacituzumab govitecan, moxetumomab pasudotox, cemiplimab, ublituximab, lampalizumab, roledumab, emapalumab, fasinumab, tanezumab, etrolizumab, NEOD001, gantenerumab, anifrolumab, tremelimumab, isatuximab, BCD-100, carotuximab, camrelizumab, IBI308, glembatumumab vedotin, mirvetuximab soravtansine, oportuzumab monatox, L19IL2/L19TNF.

Other antibodies are disclosed in International Publication No. WO2017186928, WO2018007327, WO2018031954, WO2018039247, WO2018015539, and U.S. Patent Publication No. US20180037634, US20180000935.

Other exemplary biologic agents can be FDA approved therapeutic monoclonal antibodies which include, but are not limited to, ACTEMRA® (tocilizumab, GENENTECH), ADCETRIS® (brentuximab vedotin, SEATTLE GENETICS), AMJEVITA® (adalimumab-atto, AMGEN INC), ANTHIM® (obiltoxaximab, ELUSYS THERAPEUTICS INC), ARZERRA® (ofatumumab, GLAXO GRP LTD), AVASTIN® (bevacizumab, GENENTECH), BAVENCIO® (avelumab, EMD SERONO INC), BENLYSTA® (belimumab, HUMAN GENOME SCIENCES INC.), BESPONSA® (inotuzumab ozogamicin, WYETH PHARMS INC), BLINCYTO® (blinatumomab, AMGEN), CAMPATH® (alemtuzumab, GENZYME), CIMZIA® (certolizumab pegol, UCB INC), CINQAIR® (reslizumab, TEVA RESPIRATORY LLC), COSENTYX® (secukinumab, NOVARTIS PHARMS CORP), CYLTEZO® (adalimumab-adbm, BOEHRINGER INGELHEIM), CYRAMZA® (ramucirumab, ELI LILLY AND CO), DARZALEX® (daratumumab, JANSSEN), DERMABET® (betamethasone valerate, TARO), DUPIXENT® (dupilumab, REGENERON PHARMACEUTICALS), EMPLICITI® (elotuzumab, BRISTOL MYERS SQUIBB), ENTYVIO® (vedolizumab, TAKEDA PHARMS USA), ERBITUX® (cetuximab, IMCLONE), FASENRA® (benralizumab, ASTRAZENECA AB), GAZYVA® (obinutuzumab, GENENTECH), HEMLIBRA® (emicizumab, GENENTECH INC), HERCEPTIN® (trastuzumab, GENENTECH), HUMIRA® (adalimumab, ABBVIE INC), ILARIS® (canakinumab, NOVARTIS PHARMS), ILUMYA® (tildrakizumab-asmn, MERCK SHARP DOHME), IMFINZI® (durvalumab, ASTRAZENECA UK LTD), INFLECTRA® (infliximab-dyyb, CELLTRION INC), IXIFI® (infliximab-qbtx, PFIZER INC), KADCYLA® (ado-trastuzumab emtansine, GENENTECH), KEVZARA® (sarilumab, SANOFI SYNTHELABO), KEYTRUDA® (pembrolizumab, MERCK SHARP DOHME), LARTRUVO® (olaratumab, ELI LILLY AND CO), LEMTRADA® (alemtuzumab, GENZYME), LUCENTIS® (ranibizumab, GENENTECH), MVASI® (bevacizumab-awwb, AMGEN INC), MYLOTARG® (gemtuzumab ozogamicin, WYETH PHARMS INC), MYOSCINT® (imciromab pentetate, CENTOCOR INC), NUCALA® (mepolizumab, GLAXOSMITHKLINE LLC), OCREVUS® (ocrelizumab, GENENTECH INC), OGIVRI® (trastuzumab-dkst, MYLAN GMBH), OPDIVO® (nivolumab, BRISTOL MYERS SQUIBB), PERJETA® (pertuzumab, GENENTECH), PORTRAZZA® (necitumumab, ELI LILLY CO), PRALUENT® (alirocumab, SANOFI AVENTIS), PRAXBIND® (idarucizumab, BOEHRINGER INGELHEIM), PROLIA® (denosumab, AMGEN), PROSTASCINT® (capromab pendetide, CYTOGEN), RAXIBACUMAB® (raxibacumab, HUMAN GENOME SCIENCES INC.), REMICADE® (infliximab, CENTOCOR INC), RENFLEXIS® (infliximab-abda, SAMSUNG BIOEPSIS CO LTD), REOPRO® (abciximab, CENTOCOR INC), REPATHA® (evolocumab, AMGEN INC), RITUXAN® (rituximab, GENENTECH), SILIQ® (brodalumab, VALEANT LUXEMBOURG), SIMPONI ARIA® (golimumab, JANSSEN BIOTECH), SIMULECT® (basiliximab, NOVARTIS), SOLIRIS® (eculizumab, ALEXION PHARM), STELARA® (ustekinumab, CENTOCOR ORTHO BIOTECH INC), STELARA® (ustekinumab, JANSSEN BIOTECH), SYLVANT® (siltuximab, JANSSEN BIOTECH), SYNAGIS® (palivizumab, MEDIMMUNE), TALTZ® (ixekizumab, ELI LILLY AND CO), TECENTRIQ® (atezolizumab, GENENTECH INC), TREMFYA® (guselkumab, JANSSEN BIOTECH), TROGARZO® (ibalizumab-uiyk, TAIMED BIOLOGICS USA), TYSABRI® (natalizumab, BIOGEN IDEC), UNITUXIN® (dinutuximab, UNITED THERAP), VECTIBIX® (panitumumab, AMGEN), XGEVA® (denosumab, AMGEN), XOLAIR® (omalizumab, GENENTECH), YERVOY® (ipilimumab, BRISTOL MYERS SQUIBB), ZEVALIN® (ibritumomab tiuxetan, SPECTRUM PHARMS), ZINBRYTA® (daclizumab, BIOGEN), ZINPLAVA® (bezlotoxumab, MERCK SHARP DOHME).

In some embodiments, two or more agents may be physically entrapped, encapsulated, and/or non-covalently associated with the nanostructures in the self-assembled gel. One agent may potentiate the efficacy of another encapsulated agent. Alternative, a mixture of agents (e.g., a cocktail of proteins) may be co-encapsulated to provide for continuous delivery.

Other labile proteins such as growth factors and cytokines and nucleic acids can be incorporated into the gel or co-administered with the gel for immediate release. These may be small molecules, proteins, peptides, sugars and polysaccharides, lipids and lipoproteins or lipopolysaccharides, and nucleic acids such as small interfering RNA, microRNA, PiRNA, ribozymes, and nucleotides encoding proteins or peptides. In some cases, cells can be delivered.

Non-labile compounds such as anti-inflammatory drugs, corticosteroids, local anesthetics such as lidocaine, analgesics, anti-infectious agents such as antibacterial, antifungal agents, contraceptives, and chemotherapeutics can be incorporated.

For some drugs, the loading mechanism (i.e., encapsulating and/or entrapping) is based on electrostatic interactions between the anionic amphiphile head group of an amphiphilic gelator and cationic therapeutic or prophylactic agent (i.e., drug), as can be shown when adding high salt concentrations to break these interactions and release the cationic therapeutic or prophylactic agent.

For certain types of agents such as antibodies it was found that the interaction can occur between the therapeutic or prophylactic agent (i.e., drug) and the lipophilic tails of the amphiphile gelators, which can be broken by adding a competing surfactant to release the therapeutic or prophylactic agent (i.e., drug). It was originally hypothesized that because the lipophilic regions of the self-assembled/ordered gelators were buried in the hydrogel that these lipophilic regions would be inaccessible to agent binding or loading (i.e., encapsulating and/or entrapping), but it was found that post-loading or binding of agents in a pre-formed “empty” or “unloaded” gel could be accomplished, in particular with agents as large as an antibody (up to 150 kDa).

The therapeutic or prophylactic biological agents, are generally encapsulated at a concentration between about 0.1 mg/mL and about 100 mg/mL, between about 0.1 mg/mL and about 10 mg/mL, and in other instances at a concentration of between about 0.1 mg/mL and about 5 mg/mL.

4. Gel Properties

Mechanical Property & Injectability

With self-assembled gel compositions, no gravitational flow is observed upon inversion of a container at room temperature for at least 10 seconds, and in some cases, for about 1 hour, 3 hours, 1 day, 2 days, 3 days, one week or longer. A self-assembled gel is homogeneous and stable to inversion, unlike heterogeneous materials that is a mixture of gelled regions (non-flowable) and non-gelled, liquid regions (flowable). A self-assembled gel is also different from liposome or micelle suspensions. Liposome or micelles suspensions are not self-supporting and can flow when the container is inverted.

In some embodiments, the self-assembled gel compositions have recoverable rheological properties, i.e., self-assembled gel is shear-thinning, suitable for injection, and recovers to a self-supporting state after cessation of a shear force. The self-supporting state generally features an elastic modulus of from 10 to 10,000 Pascal and greater than a viscous modulus. Due to non-covalent interactions for the assembly of gelators and cationic agents, a bulk gel may deform and be extruded under a shear force (e.g., during injection), and the gelators and cationic agents re-assemble upon cessation of shear forces to a self-supporting, stable-to-inversion state (e.g., elastic modulus G′ greater than viscous modulus G″).

Particles of the self-assembled gel composition are injectable as suspended in a pharmaceutically acceptable carrier, i.e., a suspension medium. Microparticles or nanoparticles can be formed from the bulk self-supporting gel by homogenization, sonication, or other means of dispersion in a suspension medium.

Micro- and/or Nano-Structures

The agents can be encapsulated and/or within or between the nanostructures, can be non-covalently bonded to the nanostructures, or both.

The hydrophobic parts and the hydrophilic parts of the gelator molecules interact to form nanostructures (lamellae, sheets, fibers, and/or particles) of gelator molecules. The agents can insert into and form part of the nanostructures, being encapsulated and/or entrapped in the nanostructures of the gel, or both. In hydrogels, the hydrophobic portions of gelators are located in the inner regions of a given nanostructures, and hydrophilic portions are located at the outer surfaces of the nanostructure. Several tens or hundreds of nanostructures can bundle together to form microstructures, such as fibers and sheet-like structures.

In some embodiments, the nanostructures include nanoparticles, micelles, liposome vesicles, fibers, and/or sheets. In some embodiments, the nanostructures can have a minimum dimension of 2 nm or more (e.g., 50 nm or more, 100 nm or more, 150 nm or more, 200 nm or more, 250 nm or more, 300 nm or more, 350 nm or more) and/or 400 nm or less (e.g., 350 nm or less, 300 nm or less, 250 nm or less, 200 nm or less, 150 nm or less, 100 nm or less, or 500 nm or less). In some embodiments, the nanostructures (e.g., fibers, sheets) have a length and/or width of several microns (e.g., one micron, two microns, three microns, four microns, five microns, ten microns, twenty microns, or twenty five microns) or more. The nanostructures can aggregate into networks, and/or be in the form of a liquid crystal, emulsion, fibrillar structure, or tape-like morphologies. When the nanostructures are in the form of fibers, the fibers can have a diameter of about 2 nm or more, and can have lengths of hundreds of nanometers or more. In some embodiments, the fibers can have lengths of several microns (e.g., one micron, two microns, three microns, four microns, five microns, ten microns, twenty microns, or twenty-five microns) or more.

Degradation (Cleavable Linkage)

Stimuli evoking release can be present due to the characteristics at the site of administration or where release is desired, for example, tumors or areas of infection. These may be conditions present in the blood or serum, or conditions present inside or outside the cells, tissue or organ. These are characterized by low pH and the presence of degradative enzymes. The gel compositions may be designed to disassemble only under conditions present in a disease state of a cell, tissue or organ, e.g., inflammation, thus allowing for release of an agent at targeted tissue and/or organ. This is an alternative or may be used in combination to gel erosion-mediated and passive diffusion-mediated release of agent.

This responsive release is based on linkages formed from degradable chemical bonds (or functional groups) and/or tunable non-covalent association forces (e.g., electrostatic forces, van der Waals, or hydrogen bonding forces). In some embodiments, these linkages are (1) degradable covalent linkage between the hydrophilic segment and the hydrophobic segment of an amphiphile gelator, (2) positioned in a prodrug-type gelator, which upon cleavage releases an active drug, and/or (3) covalent linkage or non-covalent association forces between a gelator and a therapeutic agent. The cleavage or dissociation of these linkages result in (1) more rapid or greater release of the encapsulated or entrapped agents compared to passive diffusion-mediated release of agent; and/or (2) converts prodrug gelator into active drug for release.

Stimuli evoking release includes intrinsic environment in vivo and user-applied stimulation, for example, enzymes, pH, oxidation, temperature, irradiation, ultrasound, metal ions, electrical stimuli, or electromagnetic stimuli. A typical responsive linkage is cleavable through enzyme and/or hydrolysis, based on a chemical bond involving an ester, an amide, an anhydride, a thioester, and/or a carbamate. In some embodiments, phosphate-based linkages can be cleaved by phosphatases or esterase. In some embodiments, labile linkages are redox cleavable and are cleaved upon reduction or oxidation (e.g., —S—S—). In some embodiments, degradable linkages can be cleaved at physiological temperatures (e.g., from 36 to 40° C., about 36° C., about 37° C., about 38° C., about 39° C., about 40° C.). For example, linkages can be cleaved by an increase in temperature. This can allow use of lower dosages, because the agents are only released at the required site. Another benefit is lowering of toxicity to other organs and tissues. In certain embodiments, stimuli can be ultrasound, temperature, pH, metal ions, light, electrical stimuli, electromagnetic stimuli, and combinations thereof.

Controlled Release

The gel compositions can be designed for controlled degradation at a site of delivery or after a period of time, based on the conditions at the site of administration. Compared to free agent in a solution, the encapsulated and/or entrapped agent releases from the self-assembled gel much slower, for example, less than 30% of encapsulated and/or entrapped agent is released in the first three days and less than 70% in seven days. In the presence of a stimulus such as an enzyme, self-assembled gel formed from a gelator with an enzyme-degradable linkage releases the agent more rapidly, compared to the gel in a medium lacking the enzyme.

The gel compositions can be prepared for controlled release and/or degradation over a period of time. Degradation may result in release of encapsulated and/or entrapped agent(s) upon cleavage of enzyme cleavable bonds in the gelators used to form the gels. The self-assembled gels may result in a cumulative release of up to about 10%, about 20%, about 30%, about 40%, about 50%, about 60%, about 70%, about 80%, about 90%, or essentially all of the agents in the gel within about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24 hours. In some instances, cumulative release of up to about 10%, about 20%, about 30%, about 40%, about 50%, about 60%, about 70%, about 80%, about 90%, or essentially all of the agents in the gel occurs within about 1, about 2, about 3, about 4, about 5, about 6, about 7, about 8, about 9, or about 10 days. In yet other instances, cumulative release of up to about 10%, about 20%, about 30%, about 40%, about 50%, about 60%, about 70%, about 80%, about 90%, or essentially all of the agents in the gel occurs within about 1, about 2, about 3, about 4, about 5, about 6, about 7, about 8 weeks or longer. In other instances, cumulative release of up to about 10%, about 20%, about 30%, about 40%, about 50%, about 60%, about 70%, about 80%, about 90%, or essentially all of the agents in the gel occurs within about 1, about 2, about 3, about 4, about 5, about 6, about 7, about 8, about 9, about 10, about 11, or about 12 months. In certain embodiments, the gels may include one or more control release agents which may increase or decrease the rate of release of the encapsulated and/or entrapped agents based on the amount of the control release agent present. An exemplary control release agent is cholesterol.

In some embodiments, the release kinetics of the drugs can be tuned by including one or more additional co-gelators, such as GRAS amphiphiles described above, which can be used to increase or decrease the rate of release of the agents encapsulated and/or entrapped within the nanostructures, such as fibers, of the gels.

Stability

The stability of the agent(s) can be determined as percentage of the activity in the gels after a certain period of time. In certain instances, the agent(s) present in gels may remain stable for at least about 1, about 2, about 3, about 4, about 5, about 6, about 7, about 8, about 9, about 10, about 11, or about 12 weeks when stored at room temperature, incubated at 4° C., or stored at 37° C. In certain other instances, agent(s) present in gels may remain stable for at least about 1, about 2, about 3, about 4, about 5, about 6, about 7, about 8, about 9, about 10, about 11, or about 12 months when stored at room temperature, incubated at 4° C., or stored at 37° C. “Remain stable,” as used herein refers to a percent loss of the agent(s) thereof of less than about 10%, about 9%, about 8%, about 7%, about 6%, about 5%, about 4%, about 3%, about 2%, about 1%, about 0.5%, or about 0.1% over a defined period of time.

5. Gel Formulations

Self-assembled gel formulations may be prepared in dry powder formulations or liquid formulations. The gels are typically sterilized or sterile. For example, a sterile formulation can be prepared by first performing sterile filtration of gelators, cationic agents, as well as agents to be encapsulated, followed by processes of preparing the gels in an aseptic environment. Alternatively, all processing steps can be performed under non-sterile conditions, and then terminal sterilization (e.g., gamma or E-beam irradiation) can be applied to the resulting hydrogels or products thereof.

Dry formulations contain lyophilized self-assembled gel compositions where solvent is removed, resulting in xerogels. Xerogels can be in a powder form, which can be useful for maintaining sterility and activity of agents during storage and for processing into desired forms. As xerogels are solvent free, they can have improved shelf-life and can be relatively easily transported and stored. To lyophilize self-assembled gels, the gels can be frozen (e.g., at −80° C.) and vacuum-dried over a period of time to provide xerogel s.

Alternatively, a dry formulation contains dry powder components of gelators, cationic agents, one or more therapeutic agents, which are stored in separate containers, or mixed at specific ratios and stored. In some embodiments, suitable aqueous and organic solvents are included in additional containers. In some embodiments, dry powder components, one or more solvents, and instructions on procedures to mix and prepare assembled nanostructures are included in a kit.

Liquid gel formulations contain self-assembled gel composition suspended in a liquid pharmaceutical carrier. In some forms, self-assembled gel is suspended or re-suspended in aqueous media for ease of administration and/or reaching a desired concentration for minimizing toxicity.

The liquid formulations may be isotonic relative to body fluids and of approximately the same pH, ranging from about pH 4.0 to about pH 8.0, more preferably from about pH 6.0 to about pH 7.6. The liquid pharmaceutical carrier can include one or more physiologically compatible buffers, such as a phosphate or bicarbonate buffers. One skilled in the art can readily determine a suitable saline content and pH for an aqueous solution that is suitable for an intended route of administration.

In some instances, the liquid formulations may include one or more suspending agents, such as cellulose derivatives, sodium alginate, polyvinylpyrrolidone, gum tragacanth, or lecithin. Liquid formulations may also include one or more preservatives, such as ethyl or n-propyl p-hydroxybenzoate.

### III. Method of Making

1. Making a Self-Assembled Gel

Generally, a water-miscible organic solvent is used to dissolve gelators to form a gelator solution. An aqueous medium (e.g., water, hypotonic solution, isotonic solution, or hypertonic solution) is added and mixed with the gelation solution. At appropriate volume ratios of the organic solvent and the aqueous solution, gelation begins as soon as the aqueous medium is mixed with the gelator solution. Over time, the gel becomes consistent. Gelation is deemed complete when the gel is self-supporting and stable to inversion at room temperature, i.e., not “runny” or flow due to gravity, and preferably having little to no precipitates and little to no aggregates therein. A self-assembled gel is homogeneous and stable to inversion, unlike heterogeneous materials that are a mix of gelled regions (non-flowable) and non-gelled, liquid regions (flowable).

Organic solvent(s) used in the gelation medium can be selected based on the solubility of gelators therein, its polarity, hydrophobicity, water-miscibility, and in some cases the acidity. Suitable organic solvents include water-miscible solvent, or solvent that has an appreciable water solubility (e.g., greater than 5 g/100 g water), e.g., DMSO, dipropylene glycol, propylene glycol, hexyl butyrate, glycerol, acetone, dimethylformamide (DMF), tetrahydrofuran, dioxane, acetonitrile, alcohol such as ethanol, methanol or isopropyl alcohol, as well as low molecular weight polyethylene glycol (e.g., 1 kD PEG which melts at 37° C.). In other forms, the self-assembled gel compositions can include a polar or non-polar solvent, such as water, benzene, toluene, carbon tetrachloride, acetonitrile, glycerol, 1,4-dioxane, dimethyl sulfoxide, ethylene glycol, methanol, chloroform, hexane, acetone, N, N′-dimethyl formamide, ethanol, isopropyl alcohol, butyl alcohol, pentyl alcohol, tetrahydrofuran, xylene, mesitylene, and/or any combination thereof. Organic solvents for gelation include dimethyl sulfoxide (DMSO), dipropylene glycol, propylene glycol, hexyl butyrate, glycerol, acetone, dimethylformamide, tetrahydrofuran, dioxane, acetonitrile, ethanol, and methanol. Another class of organic solvents, fatty alcohols or long-chain alcohols, are usually high-molecular-weight, straight-chain primary alcohols, but can also range from as few as 4-6 carbons to as many as 22-26, derived from natural fats and oils. Some commercially important fatty alcohols are lauryl, stearyl, and oleyl alcohols. Some are unsaturated and some are branched. Minimal amounts are preferred, and most if not all is removed by evaporation and/or washing following gel formation.

The aqueous solvent is typically water which may be sterilized and selected from distilled water, de-ionized water, pure or ultrapure water. In some instances the second solvent is an aqueous solution such as saline, other physiologically acceptable aqueous solutions containing salts and/or buffers, such as phosphate buffered saline (PBS), Ringer's solution, and isotonic sodium chloride, or any other aqueous solution acceptable for administration to a subject, such as an animal or human. The amounts of aqueous solvent, such as water, is typically based on the amounts of the first organic solvent used wherein the selected total volume or weight percentage of organic solvent(s) determined the volume or weight percentage of the water or aqueous solution (i.e., if 30 v/v % of organic solvent then 70 v/v % water).

In some instances, the amount of an organic solvent is no more than 1:1, 1:2, 1:3, 1:4, 1:5, 1:6, 1:7, 1:8, 1:9, 1:10, or less in volume compared to the volume of an aqueous solution (e.g., water, aqueous buffer, aqueous salt solution, optionally containing one or more additional agents). That is, the volume amount of an organic solvent in the total amount of liquid as used in forming a homogenous gel is generally less than about 50%, 33%, 25%, 20%, 17%, 14%, 12.5%, 11%, 10%, 9%, 8%, 7%, 6%, 5%, 4%, 3%, 2%, or significantly less, typically 10% or less.

Gelation may require heating the gelation medium to temperatures ranging from between about 30-100° C., about 40-100° C., about 50-100° C., about 60-100° C., about 70-100° C., about 90-100° C., about 30-90° C., about 40-90° C., about 50-90° C., about 60-90° C., about 70-90° C., about 80-90° C., about 40-80° C., about 50-80° C., about 60-80° C., about 70-80° C., about 30-70° C., about 40-70° C., about 50-70° C., about 60-70° C., about 30-60° C., about 40-60° C., about 50-60° C., about 30-50° C., or about 40-50° C. In some embodiments, heating is carried out in the temperature range of between about 60-80° C. In some embodiments, the heating is carried out at about 80° C. In all cases, the gel or gel initiated reagents are cooled to body temperature or less when labile agent is incorporated.

In some instances, no heating is needed, or, if necessary, heating to about body temperature (37° C.) generates a homogeneous self-supporting gel that is stable to inversion. In other embodiments, the gelation medium is heated to complete dissolution, followed by cooling to about 37° C. or room temperature around 20° C.-25° C.

Gelation can take place with or without heating. When heated, gelation can take place as the heated gelation solution is cooled. Leaving the gel on a stable surface for about one to two hours at room temperature results in a consistent self-supporting gel. Self-supporting gel comprises orderly assembled micro- or nano-structures with minimal precipitates. This is generally confirmed using optical or electron microscopy.

2. Loading Self-Assembled Gel with Agent(s)

In preferred embodiments, the agent(s) may be physically entrapped, encapsulated, and/or non-covalently associated with the nanostructures of the self-assembled gels by first forming the gel and then suspending the gel in an aqueous medium, such as a buffer, where the gel is optionally first broken to form particles (i.e., nano- and/or microparticles). Preferably, the self-assembled gel formed is free of or substantially free of organic solvent(s). Subsequently, the resulting gel suspension, which may be a gel particle suspension, is mixed with a second solution or suspension containing one or more agent(s) described herein. Typically the second solution or suspension is a buffer solution containing agent(s). Mixing may be carried out by any appropriate means. Non-limiting mixing means include pipetting and/or vortexing. Mixing may be carried out at room temperature. In some instances, no heating is needed when mixing.

In some forms, the bulk self-assembled gel prior to agent(s) being loaded is first suspended in water, phosphate buffered saline, or other physiological saline, which is homogenized or sonicated to break up the bulk gel into particles which retain the fibrous nanostructures formed in the bulk gel. These particles may be collected, stored, purified, and reconstituted prior to loading of agent(s). Different types of gel particles may be loaded with different amounts or types of agents.

In a non-limiting example, a method of forming a self-assembled gel and loading the gel composition with agent(s), such as biologics, can include the steps of:
(a) forming a solution comprising a gelator having a molecular weight of 2,500 or less in a medium comprising water or an aqueous solution and optionally an organic solvent;
(b) optionally heating the solution and then cooling the solution to afford a self-assembled gel;
(c) optionally removing all or substantially all of the organic solvent, when present, from the self-assembled gel;
(d) suspending the self-assembled gel in water, a phosphate buffered saline, or some other physiological saline, and optionally wherein the self-assembled gel is homogenized or sonicated to break up the self-assembled gel into particles (such as microparticles and/or nanoparticles);
(e) providing a solution or suspension of one or more agents (such as therapeutic, prophylactic, and/or biologic agents) in water, a phosphate buffered saline, or some other physiological saline; and
(f) mixing the self-assembled gel suspension and agent-containing suspension to load the biologic agent into the self-assembled gel.

In another non-limiting example, the method of forming a self-assembled gel and loading the gel composition with agent(s), such as biologics, can include the steps of:
(a′) forming a solution containing a gelator having a molecular weight of 2,500 or less in a medium containing water or an aqueous solution and optionally an organic solvent;
(b′) optionally heating the solution and then cooling the solution to afford a self-assembled gel;
(c′) suspending the self-assembled gel in water, a phosphate buffered saline, or some other physiological saline, optionally wherein the self-assembled gel is homogenized or sonicated to break up the self-assembled gel into particles (such as microparticles and/or nanoparticles);
(d′) optionally removing all or substantially all of the organic solvent, when present from the suspended self-assembled gel, such as by centrifugation, tangential flow filtration, evaporation, or other suitable means;
(e′) providing a solution or suspension of one or more agents such as therapeutic, prophylactic, and/or diagnostic agents in water, a phosphate buffered saline, or some other physiological saline; and
(f′) mixing the self-assembled gel suspension and agent-containing suspension to load the biologic agent into the self-assembled gel.

In yet another non-limiting example, the method of forming a self-assembled gel and loading the gel composition with agent(s), such as biologics, can include the steps of:
(a″) forming a solution comprising a gelator having a molecular weight of 2,500 or less in a medium containing water or an aqueous solution and optionally an organic solvent;
(b″) optionally heating the solution and then cooling the solution to afford a self-assembled gel;
(c″) suspending the self-assembled gel in water, a phosphate buffered saline, or some other physiological saline, optionally wherein the self-assembled gel is homogenized or sonicated to break up the self-assembled gel into particles (such as microparticles and/or nanoparticles);
(d″) providing a solution or suspension of one or more agents (such as therapeutic, prophylactic, and/or biologic agents) in water, a phosphate buffered saline, or some other physiological saline;
(e″) mixing the self-assembled gel suspension and agent-containing suspension to load the biologic agent into the self-assembled gel; and
(f″) optionally, removing all or substantially all of the organic solvent, when present, from the self-assembled gel and optionally removing any excess non-encapsulated and/or non-entrapped agent(s) such as by washing or other purification means.

When organic solvent is used the organic solvent is preferably removed entirely or substantially, typically by a lyophilization or a drying step. Removal of the organic solvent from the resulting self-assembled gel may be complete or a substantial removal of organic solvent(s) thereof. Substantial removal refers to less than about 5%, 4%, 3%, 2%, 1%, 0.9%, 0.8%, 0.7%, 0.6%, 0.5%, 0.4%, 0.3%, 0.2%, 0.1% or less of organic solvent by weight of the resulting self-assembled gel.

In some instances, the organic solvent(s) used in formation of the gel are not removed but instead the concentration of organic solvent(s) is reduced by dilution with a sufficient amount of water, a phosphate buffered saline, or some other physiological saline. In a non-limiting example, the resulting self-assembled gel containing organic solvent(s) is suspended in water, a phosphate buffered saline, or some other physiological saline and the amount of organic solvent(s) is diluted by the added water, a phosphate buffered saline, or some other physiological saline such that the effective concentration of organic solvent(s) in the suspension is less than about 5%, 4%, 3%, 2%, 1.5%, 1%, 0.9%, 0.8%, 0.7%, 0.6%, 0.5%, 0.4%, 0.3%, 0.2%, 0.1% or less by volume of the suspension. In some instances, the effective concentration of organic solvent(s) in the suspension is between about 2 and about 4% by volume of the suspension. In methods where water, a phosphate buffered saline, or some other physiological saline is added for purposes of diluting the amount of organic solvent(s) the amount of organic solvent following dilution is typically no greater than 5%, 10%, 15%, or 20% by volume.

The amount of the self-assembled gel suspended in the water, a phosphate buffered saline, or some other physiological saline is about 1, 2, 3, 4, 5, 6, 7, 8, 9, 10, 15, 20, 25, 30, 35, 40, 45, 50, 60 or 70 mg/mL. In some instances, the amount of the self-assembled gel suspended in the water, a phosphate buffered saline, or some other physiological saline is between about 10 and about 20 mg/mL.

The amount of the agent(s) dissolved or suspended in the water, a phosphate buffered saline, or some other physiological saline is about 0.1, 1, 2, 3, 4, 5, 6, 7, 8, 9, 10, 15, 20, 25, 30, 35, 40, 45, or 50 mg/mL. The preferred range is 10 mg/ml or less.

Suspension of the self-assembled gel in water, a phosphate buffered saline, or some other physiological saline or suspension of agent(s) in water, a phosphate buffered saline, or some other physiological saline may be carried out by stirring, agitation, vortexing, or any other suitable method.

In some embodiments, particles are nanoparticles having a hydrodynamic diameter between 100 nm and 990 nm, preferably between 500 nm and 900 nm, and the nanoparticles maintain at least 50, 60, 70 or 80% of the size in serum over a period of at least two hours. In other embodiments, particles are microparticles having a diameter ranging from 1 μm to a couple hundred millimeters. Particles can have sizes within the range of about 0.1-3000 microns, more preferably about 0.5-1000 microns, and larger particles and/or aggregates thereof can be optionally broken to reduce the size to a range of about 0.5-200 microns. In some embodiments, the nanoparticles and/or microparticles have a minimum dimension of 2 nm or more, 50 nm or more, 100 nm or more, 150 nm or more, 200 nm or more, 250 nm or more, 300 nm or more, 350 nm or more, 500 nm or more, 1,000 nm or more, 5,000 nm or more, or 10,000 nm or more) and/or 400 nm or less (e.g., 10,000 nm or less, 5,000 nm or less, 1,000 nm or less, 500 nm or less, 350 nm or less, 300 nm or less, 250 nm or less, 200 nm or less, 150 nm or less, 100 nm or less, or 500 nm or less). The particles may aggregate into networks, and/or be in the form of a liquid crystal, emulsion, or other types of morphologies.

Preferably, loading of the agent(s) into the gel occurs without or with only minimal exposure of the agent(s) to organic solvent(s) which may degrade or destroy the agents or their activity. Preferably, loading of the agent(s) into the self-assembled gel occurs without or with only minimal exposure of the agent(s) to heating temperatures which may degrade or destroy the agents. Typically, the agent(s) encapsulated and/or entrapped in the gel and structures therein remain stable within the gel and retain at least about 50%, 60%, 70%, 80%, 90%, 95%, or greater up to essentially 100% of their activity upon release from the gel, as compared to the activity of the agent prior to loading in the gel.

The gels demonstrate drug loading efficiencies of the one or more agents up to about 50 wt/wt %, about 45 wt/wt %, about 40 wt/wt %, about 35 wt/wt %, about 30 wt/wt %, about 25 wt/wt %, about 20 wt/wt %, about 15 wt/wt %, about 10 wt/wt %, or about 5 wt/wt % of the agent(s) to gel. The methods permit high loading efficiencies of the one or more agents into the gel (by post-loading the pre-formed gels) which is higher than the loading efficiency of same one or more agents when loaded into the gelation medium prior to the formation of a self-assembled gel.

The gels may demonstrate encapsulation efficiencies of the one or more agents up to about 100 wt/wt %, 99 wt/wt %, 98 wt/wt %, 97 wt/wt %, 96 wt/wt %, 95 wt/wt %, 94 wt/wt %, 93 wt/wt %, 92 wt/wt %, 91 wt/wt %, 90 wt/wt %, about 80 wt/wt %, about 70 wt/wt %, about 60 wt/wt %, about 50 wt/wt %, about 45 wt/wt %, about 40 wt/wt %, about 35 wt/wt %, about 30 wt/wt %, about 25 wt/wt %, about 20 wt/wt %, about 15 wt/wt %, about 10 wt/wt %, or about 5 wt/wt %.

Agent loading level can be time-dependent and the loading levels in the above methods may be controlled and/or optimized by way of the loading/incubation time in any of the above mixing steps described. In some instances, the loading/incubation time may be selected from a period of time ranging from between about 0.1 and 48 hours, 0.1 and 36 hours, 0.1 and 24 hours, 0.1 and 20 hours, 0.1 and 15 hours, 0.1 and 10 hours, 0.1 and 5 hours, 0.1 and 1 hours, or ranges therein. In some other instances, the loading/incubation time may be selected from a period of time of at least about 0.1 hours, 0.2 hours, 0.3 hours, 0.4 hours, 0.5 hours, 0.6 hours, 0.7 hours, 0.8 hours, 0.9 hours, 1 hours, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or greater.

Agent loading level can be pH and/or charge-dependent and the loading may be controlled and/or optimized by way controlling the pH during any of the mixing steps described above. In some instances, the pH of one or both of the gel suspension or agent(s)-containing solution or suspension are modified to a desired pH and then mixed. In some other instances, the gel suspension or agent(s)-containing solution or suspension are mixed and the pH of the mixture formed is modified as desired. Agent loading and encapsulation efficiency may be optimized/maximized by loading gel suspensions described at a pH above the pKa of the gelator(s) and a pH which is below the isoelectric (pI) point of the agent(s) which are being loaded onto the gel. The pKa's and pI's of the gelators(s) and agent(s) are either known art known values or may be determined using known techniques.

The preferred pH range is 4.4 to 8.7.

In preferred embodiments, the methods described above show greater encapsulation efficiency of agent(s) due to the self-assembly process of the gelators into microfibers, which is believed to be due to greater surface area available for gelator-agent interaction. This is in contrast to the use of non-self assembled gelator particulate suspensions, as discussed in the Examples below.

The self-assembled gels loaded with agent(s) in some embodiments are suspended in a pharmaceutically acceptable for ease of administration to a patient (e.g., by drinking or injection) and/or to provide a desired drug concentration to control toxicity.

3. Gel Purification

Distillation, filtration, dialysis, centrifugation, tangential flow filtration, evaporation, other solvent exchange techniques, vacuum drying, or lyophilization may be used in one or more repeated processes to remove organic solvent(s) and/or unencapsulated and/or unentrapped excess agent(s) or any other unencapsulated and/or unentrapped agents present from the gels to below the stated limit of pharmaceutical product requirements. Solvent removal and/or removal of unencapsulated and/or unentrapped agent(s) can be carried out on the gel directly following formation, following formation of the gel suspension, or after the agent(s) has been loaded into the gel suspension.

Generally a purification (wash) medium is one suitable for administration, such that the solvent of the gel is at least partially replaced with the purification medium.

Generally, a process to make the self-assembled gel composition includes combining gelators, cationic agents, and solvents to form a mixture; heating and/or sonicating the mixture; stirring or shaking the mixture for a time sufficient to form a homogeneous solution; and cooling the homogenous solution for a time sufficient to enable the formation of self-assembled gel compositions.

4. Sterilization

A sterile formulation is prepared by first performing sterile filtration of the process solutions (e.g., drug and gelator solutions), followed by gel preparation, suspension, purification and lyophilization under aseptic procession conditions. Alternatively, all processing steps can be performed under non-sterile conditions, and then terminal sterilization (e.g., gamma or E-beam irradiation) can be applied to the lyophilized hydrogel product. Sterile solution for resuspension can also be prepared using similar methods.

### IV. Methods of Use

The gel composition, the fibrous suspension, or the gel particle suspension, can be administered through various known regional delivery techniques, including injection, implantation, topical application to the mucosa, such as the oral or buccal surfaces, nasal or pulmonary tracts, intestinal tracts (orally or rectally), vagina, or skin. In situ self-assembly of stabilized nanostructures allows for regional delivery of the compositions and stimuli-responsive delivery of active agents, especially to areas of infection, trauma, inflammation or cancer.

Delivered agent(s) can be controllably released from the gel compositions in response to stimuli for targeted release. In the absence of stimuli, the agent is released in a sustained manner with little to no burst release. For example, encapsulated agents can be gradually released over a period of time (e.g., hours, one day, two days, three days, a week, a month, or more). Depending on the parameters, release can be delayed or extended from minutes to days to months, for example, when gel compositions are administered under physiological conditions (a pH of about 7.4 and a temperature of about 37° C.).

For example, parenteral administration includes administration to a patient intradermally, intraperitoneally, intramuscularly, subcutaneously, subjunctivally, by injection.

The compositions are useful for improving targeting efficiency, efficacy, safety, and compliance benefiting from single dose, prolonged action or tissue-specific formulations, compared to agents delivered in its free solution form. In some embodiments, the compositions can be useful to release agents that correlate with different stages of tissue regeneration.

Exemplary diseases or disorders to be treated with the assembled nanostructures include, but are not limited to, allergy (e.g. contact dermatitis), arthritis, asthma, cancer, cardiovascular disease, diabetic ulcers, eczema, infections, inflammation, periodontal disease, psoriasis, respiratory pathway diseases (e.g., tuberculosis), vascular occlusion, pain, graft versus host diseases, canker sores, mucositis, inflammatory bowel disease including Crohn's disease and ulcerative colitis, ulcerative proctitis, pouchitis, esophagitis, interstitial cystitis, uveitis, rhinitis, bacterial conditions, viral conditions.

In some forms, the self-assembled gel composition is used in a method of preventing or treating one or more symptoms any one of the exemplary diseases or disorders in a subject by administering an effective amount of the self-assembled gel composition to deliver an effective amount of the agent(s).

The present invention will be further understood by reference to the following non-limiting examples.

### Example 1: Loading, Release, and Activity of Infliximab and Adalimumab in Ascorbyl Palmitate (AP) Gels

Methods:

Preparation of AP Gel Microparticle Suspension:

A 10 mg/mL ascorbyl palmitate (AP) microparticle suspension was prepared in a 20 mL vial with stir bar by dissolving 200 mg of AP in 700 μL DMSO. 2.8 mL of water was added and the vial was placed in a 80° C. water bath with stirring at 240 rpm for 6 minutes. Subsequently, the mixture in the vial was cooled in a room temperature water bath overnight to form a gel. Lastly, 17.5 mL of PBS buffer was added to the mixture and agitated to suspend gel.

Loading AP Gel Microparticle Suspension with Monoclonal Antibody (mAb):

500 μL of the 10 mg/mL AP suspension prepared above was added to a microcentrifuge tube. 500 μL of 2 mg/mL of infliximab (IFX) or adalimumab (ADA) in PBS buffer was added to the tube. Mixing was performed by gentle pipetting or by gentle vortexing to form an IFX loaded AP Gel Microparticle Suspension (IFX/AP) and an ADA loaded AP Gel Microparticle Suspension (ADA/AP). IFX and ADA control was prepared for purposes of loading assessment by repeat the preceding step but instead using 500 μL of PBS buffer in place of the AP gel microparticle suspension.

Measuring Loading of Monoclonal Antibody (mAb):

The IFX/AP, ADA/AP, IFX control, and ADA control samples were centrifuged at 20,000×g for 5 minutes. Each supernatant was assayed for total protein using the Coomassie plus assay (following the kit instructions) or a calibrated HPLC method. The difference in mAb content between the IFX control and IFX/AP sample, as well as the ADA control and ADA/AP sample, corresponded to the amount of mAb loaded in the respective AP gels.

Measuring mAb Release from IFX/AP Gel and ADA/AP Gel Samples:

In a centrifuge tube, 500 μL of the IFX/AP gel sample or ADA/AP gel sample were mixed with 500 μL of fasted state simulated intestinal fluid (FaSSIF; BioRelevant) containing either 0 or 50 μg/mL lipase (Sigma-Aldrich L0777). At selected time points, samples were taken from each respective gel and centrifuged at 5,000×g for 5 minutes and a small sample was removed for mAb quantification via Coomassie plus or HPLC.

Measuring Activity of mAb Released from IFX/AP Gel and ADA/AP Gel Samples:

Activity was measured using the TNF-α activity assay with fibroblast L929 cells (Gibco cytotoxicity assay for recombinant proteins).

### Results

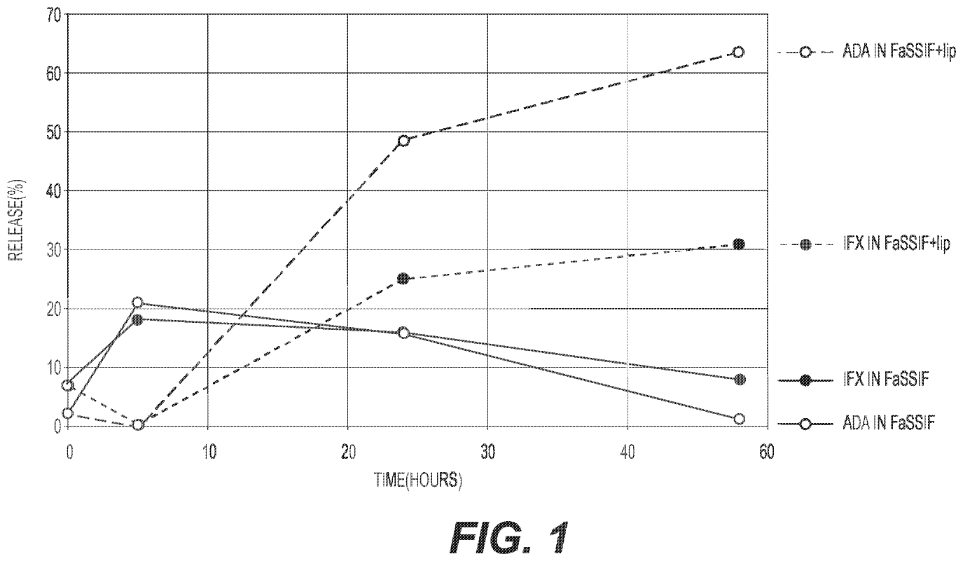

Loading and release of infliximab (IFX) & adalimumab (ADA) from the loaded AP gels were studied. As shown in FIG. 1, both IFX and ADA antibodies were shown to have >90% encapsulation efficiency (time 0 measurement). Based on percent encapsulated, it was calculated that there was ˜15-20 wt % mAb loading of the gels.

ADA/AP and IFX/AP gels were responsive to lipase in FaSSIF, as shown by higher percent free mAb at 24 and 48 hours in samples containing lipase versus lipase-free FaSSIF.

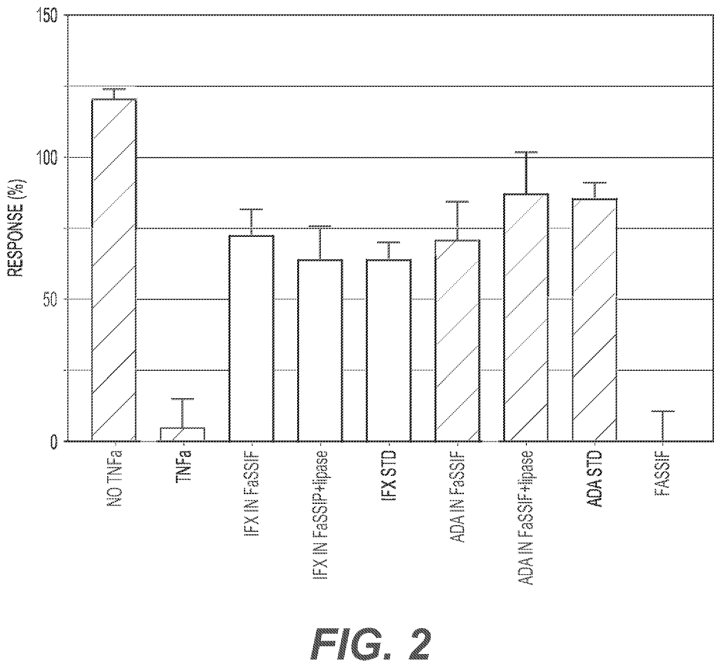

As shown in FIG. 2, both the IFX and ADA released from the gels were active against TNF-α in the L929 viability assay, as shown by similar responses compared to IFX and ADA controls.

### Example 2: Time-Dependent Loading of Infliximab and Adalimumab in Ascorbyl Palmitate (AP) Gels

Time dependent loading optimization of antibody (i.e., IFX or ADA) into ascorbyl palmitate microfiber suspensions was tested by preparing IFX/AP gel and ADA/AP gel samples according to the methods described in Example 1.

Results

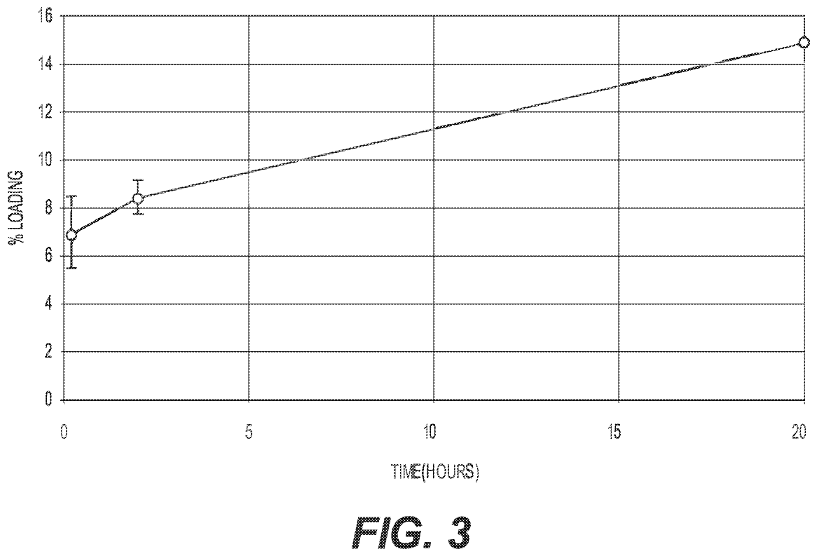

The efficiency of antibody (i.e., IFX or ADA) loading into ascorbyl palmitate microfibers was found be time dependent. As shown in FIG. 3, the infliximab (IFX) loading is 7% (wt/wt %) after 10 minutes of loading/incubation at 22° C. (and following 3 washes with PBS by centrifugation at 5000 rpm for 10 mins at 4° C.). However, by extending the loading/incubation time to 20 hours (2 hours at 22° C., 18 hours at 4° C.), the loading increased to 14% (wt/wt %). The theoretical maximum loading was calculated to be approximately 16.7% (wt/wt %) based on the amounts of infliximab (IFX) and AP combined during the loading process, which equates to loading efficiency of about 84%.

### Example 3: pH and/or Charge-Dependent Loading of Infliximab and Adalimumab in Ascorbyl Palmitate (AP) Gels

pH and/or charge-dependent loading optimization of antibody (i.e., IFX or ADA) into ascorbyl palmitate microfiber suspensions were tested by preparing IFX/AP gel and ADA/AP gel samples according to the methods described in Example 1.

Results

Due to their charged nature, antibodies such IFX and ADA have a pH dependent interaction with the acidic headgroups of ascorbyl palmitate (AP). Above its pKa of 4.4, AP has a net negative charge. Both infliximab (IFX) and adalimumab (ADA) have an isoelectric point (pI) around 8.2-8.7. The influence of pH on encapsulation efficiency is shown in FIG. 4. The highest loading and encapsulation efficiencies are observed at a pH above the pKa of AP but below the pI of IFX or ADA, which indicates that electrostatic interactions are a contributing mechanism to loading efficiency.

### Example 4: High Encapsulation Efficiency of Antibodies into Self-Assembled Ascorbyl Palmitate (AP) Microfibers, as Compared to Non-Assembled Ascorbyl Palmitate Suspensions

Materials & Methods

Ascorbyl palmitate (AP) microfiber suspensions were prepared using the self-assembly process described in Example 1 or, alternatively, using a non-assembled AP particulate used as-is from the manufacturer (USP grade ascorbyl palmitate, Sigma-Aldrich). Both the self-assembled and non-self-assembled suspensions, at 10 mg/mL, were mixed with equal volumes of either IFX or ADA at 2 mg/mL in PBS buffer. After loading/incubation at 22° C. for 2 hours, the suspensions were centrifuged at 20,000×g for 10 minutes at 4° C. The supernatant was then removed and tested for soluble protein via the Coomassie Assay to determine the encapsulation efficiency.

Results

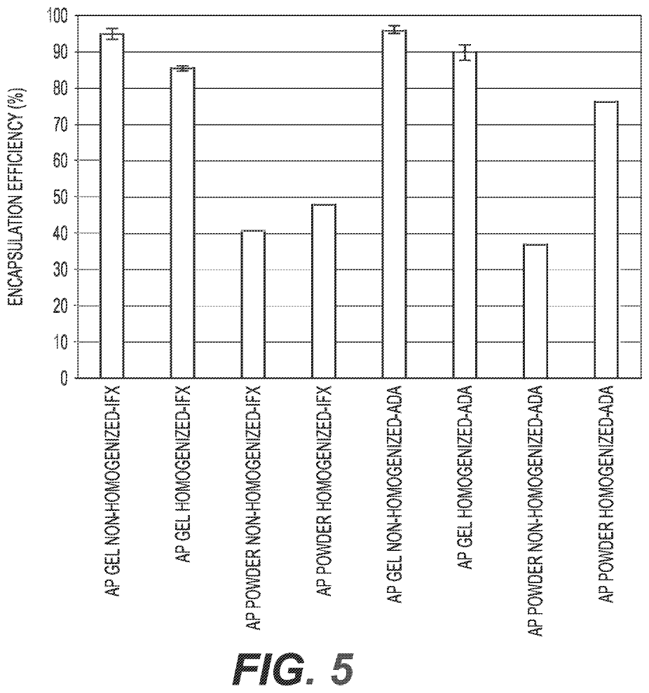

As shown in FIG. 5, the self-assembled AP microfiber suspensions resulted in greater encapsulation of antibody (IFX and ADA) relative to a suspension of non-self-assembled AP powder. When the AP suspensions (either self-assembled microfibers or non-assembled particulate) were homogenized for 1 minute, there was greater encapsulation of antibody, presumably due to greater surface area availability, resulting from the AP-antibody interaction in the self-assembled AP microfiber suspension. This data demonstrates that the self-assembly process of the AP microfibers improves the antibody encapsulation efficiency.

### Example 5: Encapsulation of Dye-Labeled IgG Antibody

Materials and Methods

FITC-IgG (Sigma Aldrich) was encapsulated and purified using the method described in Example 1. Microscopy images of antibody-loaded microfiber suspensions were taken with an EVOS FL2 Auto microscope at 40× magnification.

Results

The microscopy images demonstrated co-localization of the fluorescent antibody and ascorbyl palmitate (AP) microfibers in overlaid images. As a control, AP microfibers with no FITC-IgG tested did not demonstrate auto-fluorescence with the green fluorescent protein (GFP) filter.

Unless defined otherwise, all technical and scientific terms used herein have the same meanings as commonly understood by one of skill in the art to which the disclosed invention belongs. Publications cited herein and the materials for which they are cited are specifically incorporated by reference. Those skilled in the art will recognize, or be able to ascertain using no more than routine experimentation, many equivalents to the specific embodiments of the invention described herein. Such equivalents are intended to be encompassed by the following claim.

1-34. (canceled)

35. A method of forming a gel composite comprising a biologic agent, the method comprising:
obtaining a self-assembled gel derived from an amphiphilic gelator; and
mixing the biologic agent with the self-assembled gel for a time sufficient to load the biologic agent into the self-assembled gel.

36. The method of claim 35, wherein the self-assembled gel is obtained by the process comprising:
forming a gel precursor mixture by dissolving the amphiphilic gelator in an aqueous medium; and
forming the self-assembled gel derived from the amphiphilic gelator in the gel precursor mixture.

37. The method of claim 36, wherein the aqueous medium comprises a water-miscible organic solvent.

38. The method of claim 37, further comprising removing the organic solvent from the self-assembled gel.

39. The method of claim 36, wherein forming the self-assembled gel comprises:
heating the gel precursor mixture to a temperature sufficient to dissolve the amphiphilic gelator in the aqueous medium; and
cooling the heated gel precursor mixture, wherein, during cooling, the amphiphilic gelator self-assembles into the self-assembled gel.

40. The method of claim 35, wherein the self-assembled gel is obtained by the process comprising:
forming a gel precursor mixture by dissolving the amphiphilic gelator in a water-miscible organic solvent;
adding a sufficient amount of water to the gel precursor mixture to induce self-assembly of the amphiphilic gelator into the self-assembled gel.

41. The method of claim 35, further comprising forming particles of the self-assembled gel prior to mixing the biologic agent with the self-assembled gel.

42. The method of claim 41, wherein forming the particles comprises suspending the hydrogel in an aqueous medium and breaking the hydrogel into the particles.

43. The method of claim 41, wherein the particles comprise microparticles and/or nanoparticles.

44. The method of claim 35, further comprising drying the gel composite.

45. The method of claim 35, wherein the amphiphilic gelator comprises an ascorbyl alkanoate.

46. The method of claim 35, wherein the amphiphilic gelator comprises a sucrose alkanoate.

47. The method of claim 35, wherein the amphiphilic gelator comprises a sorbitan alkanoate.

48. The method of claim 35, wherein the amphiphilic gelator comprises ascorbyl palmitate.

49. The method of claim 35, wherein the biological agent is a protein.

50. The method of claim 35, wherein the biological agent is an antibody.

51. The method of claim 35, wherein mixing is performed without heating above 37° C.

52. The method of claim 35, wherein the self-assembled gel is free or substantially free of an organic solvent.

53. The method of claim 35, wherein the biological agent is not exposed to an organic solvent in the mixing step.

54. The method of claim 35, wherein the amphiphilic gelator has a molecular weight of 2,500 or less.

55. The method of claim 35, wherein mixing is performed for a time sufficient to achieve a drug loading up to about 50% (wt/wt).

56. The method of claim 35, wherein the loading efficiency of the biological agent is from about 70% up to about 95%.

57. The method of claim 35, wherein mixing is performed at a pH above a pKa of the amphiphilic gelator and below an isoelectric point of the biological agent.

58. The method of claim 35, wherein mixing is performed for a time of about 0.1 hour to about 48 hours.

59. The method of claim 35, wherein the concentration of the biological agent during mixing is about 0.1 mg/mL to about 100 mg/mL.
